(19) United States
(12) Patent Application Publication
(10) Pub. No.: US 2008/0234819 A1
(43) Pub. Date: Sep. 25, 2008

(54) ALL-INSIDE DOUBLE-BUNDLE ACL RECONSTRUCTION

(76) Inventors: Reinhold Schmieding, Naples, FL (US); Jacob Jolly, Naples, FL (US)

Correspondence Address: DICKSTEIN SHAPIRO LLP, 1825 EYE STREET NW, Washington, DC, 20006-5403, US

(21) Appl. No.: 12/032,566

(22) Filed: Feb. 15, 2008

Related U.S. Application Data
(60) Provisional application No. 60/901,313, filed on Feb. 15, 2007.

(51) Int. Cl. A61F 2/08; A61B 17/04; A61B 17/00

(52) U.S. Cl. 623/13.14; 606/232; 606/80

(57) ABSTRACT

An “all-inside double-bundle” ACL reconstruction technique, according to which two femoral sockets and two closed tibial sockets are provided to accommodate retrograde fixation of two grafts (for example, two semitendonosus allografts) within the four sockets. At least one of the tibial sockets is formed by using a retrograde drill device provided with a retrograde drill cutter detachable from a retrograde drill guide pin. The femoral tunnels or sockets may be formed by the retrograde drill method or by a conventional method, and may be carried out before or after the formation of the tibial sockets. The grafts (for example, two semitendonosus allografts) are secured in the knee by employing a continuous loop/button construct provided with a button, preferably of titanium alloy, and a continuous loop of suture attached to the button.

### CROSS REFERENCE TO RELATED APPLICATIONS

This application claims the benefit of U.S. Provisional Application Ser. No. 60/901,313 filed on Feb. 15, 2007, the entire disclosure of which is incorporated by reference in its entirety herein.

### FIELD OF THE INVENTION

The present invention relates to the field of surgery and, more particularly, to a method and apparatus for improved graft fixation in ACL reconstructive surgeries.

### BACKGROUND OF THE INVENTION

Reconstructive surgeries, particularly anterior cruciate ligament (ACL) reconstruction, are well-known in the art. In general, these methods of tenodesis involve drilling a tunnel through the tibia, drilling a closed tunnel (socket) into the femur, inserting a substitute ACL graft into the tunnels, and securing the grafts to the walls of the tibial and femoral tunnels using interference screws or the like.

Recently, a “double bundle” ACL technique has been developed, wherein two femoral tunnels and two tibial tunnels are drilled through femur and tibia, respectively, to allow fixation of two grafts (for example, two semitendonosus allografts) within the four tunnels. Although the “double bundle” ACL technique appears to have minimal fixation failure, the technique is nevertheless complex and difficult to reproduce as four tunnels need to be drilled to accommodate two substitute grafts and four corresponding fixation devices (for example, interference screws or tenodesis screws). The formation of four tunnels also requires large incisions and unwanted bone removal, which could ultimately result in bone fragmentation and graft failure. The formation of two converging tunnels in tibia raises additional problems, as tibia has great variations in bone density and cannot withstand interference screws at the distal end of tibial tunnels, posing a greater risk for tibial screw cyst formation. The interference screws at the distal end of the tibial tunnels may also turn, resulting in failed interference screw fixation and increased movement of the graft from side to side (decreasing the life of the ACL repair).

### BRIEF SUMMARY OF THE INVENTION

The present invention provides a technique and reconstruction system for ACL repair by providing improved fixation of the graft to be secured within the tibial and femoral tunnels.

The invention provides an “all-inside double-bundle” ACL technique, according to which two femoral sockets and two closed tibial sockets are provided to accommodate retrograde fixation of two grafts (for example, two semitendonosus allografts) within the four sockets.

Other features and advantages of the present invention will become apparent from the following description of the invention which refers to the accompanying drawings.

### BRIEF DESCRIPTION OF THE DRAWINGS

FIGS. 1-13 illustrate various steps of a method of ACL reconstruction employing an “all-inside double-bundle” technique of the present invention.

FIGS. 14 and 15 illustrate a continuous loop/button construct used for fixating a graft according to the “all-inside double-bundle” ACL technique of present invention.

### DETAILED DESCRIPTION OF THE INVENTION

The present invention provides a technique and reconstruction system for ligament or tendon repair that eliminates the formation of tibial tunnels and provides a simpler ACL reconstruction technique. The method allows proper tensioning and fixation of grafts in an “all-inside double-bundle” ACL technique, wherein formation of tibial tunnels is eliminated and graft fixation is improved.

The invention provides an “all-inside double-bundle” ACL technique, according to which two femoral sockets and two closed tibial sockets are provided to accommodate retrograde fixation of two grafts (for example, two semitendonosus allografts) within the four sockets. At least one of the tibial sockets is formed by using a retrograde drill device provided with a retrograde drill cutter detachable from a threaded guide pin, in the manner described in U.S. Patent Application Publication No. 2004/0199166, the disclosure of which is hereby incorporated by reference herein in its entirety. The femoral tunnels or sockets may be formed by the retrograde drill method or by a conventional method, and may be carried out before or after the formation of the tibial sockets.

Preparation of the grafts (for example, two semitendonosus allografts) may be conducted by employing a continuous loop/button construct provided with a button, preferably of titanium alloy, and a continuous loop attached to the button. The button has an oblong configuration and a width that is preferably less than about 1 mm narrower than the width of the drill hole through which the button is inserted and subsequently passed through. The button is provided with an inside eyelet that allows the passage of the continuous loop.

The invention also provides a method of ACL reconstruction using two grafts provided intraarticularly within two tibial sockets and two femoral sockets. The method of ACL reconstruction comprises, for example, the steps of: (i) drilling two tibial tunnels or sockets using a retrograde drill technique; (ii) drilling two femoral tunnels or sockets; (iii) providing two grafts (soft tissue grafts or BTB grafts) in the vicinity of the two femoral and two tibial sockets; and (iv) securing the two grafts within the tibial sockets by employing a continuous loop/button construct for improved fixation of graft to bone.

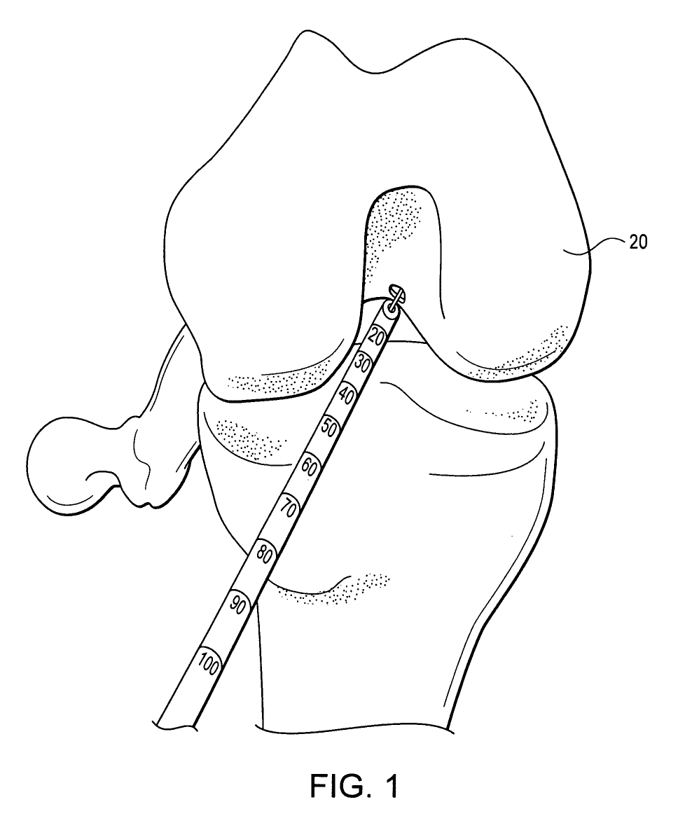

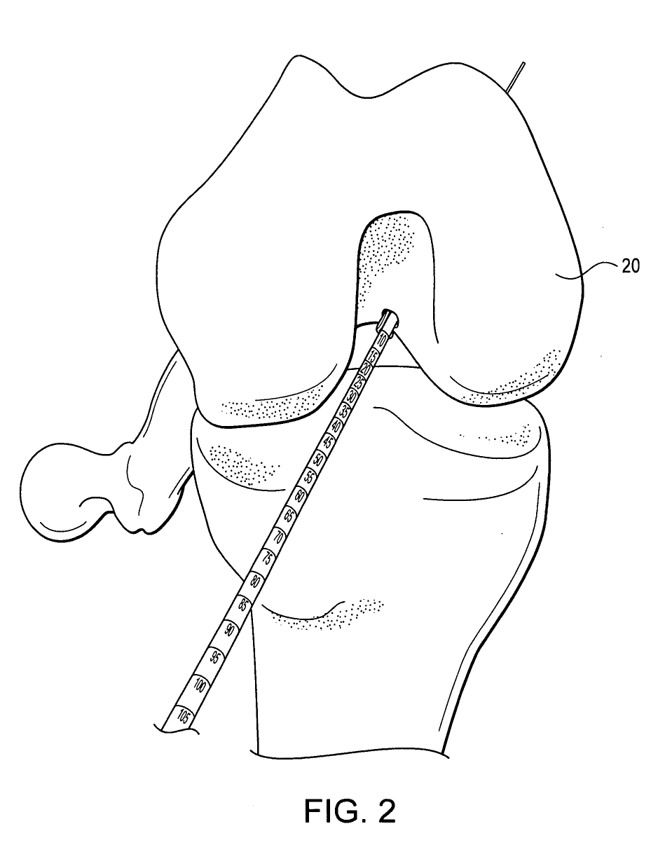

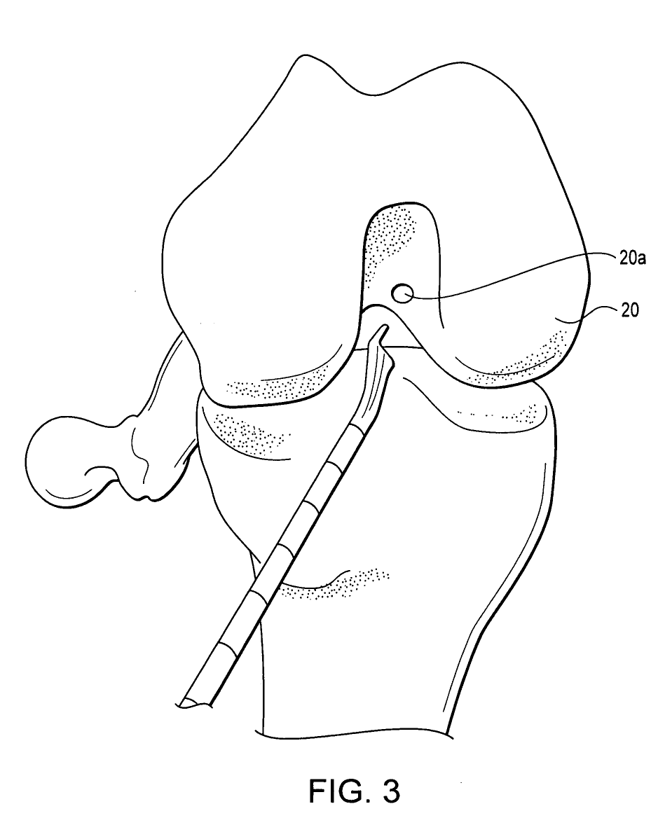

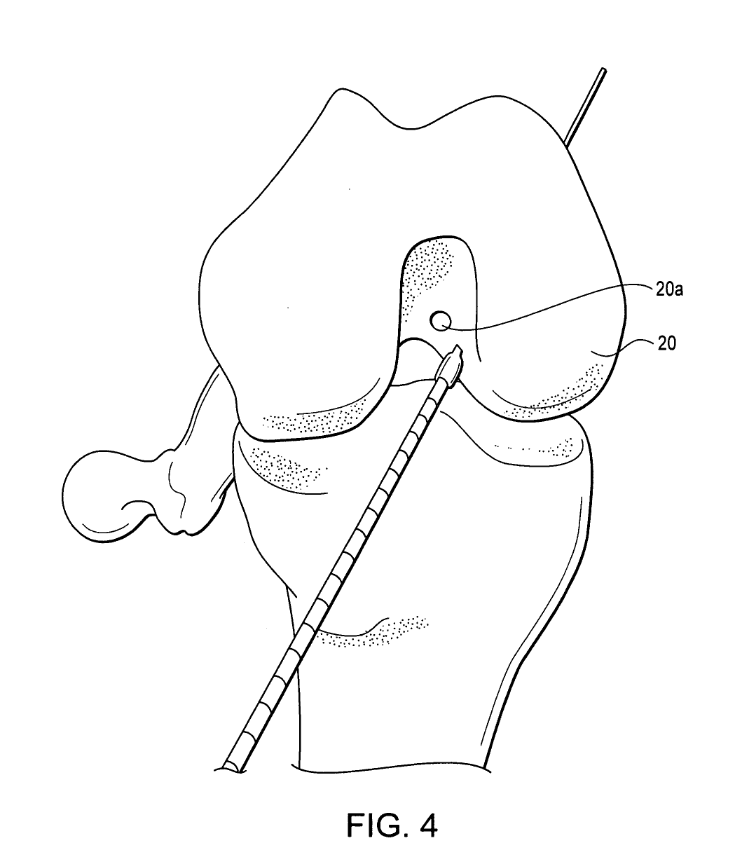

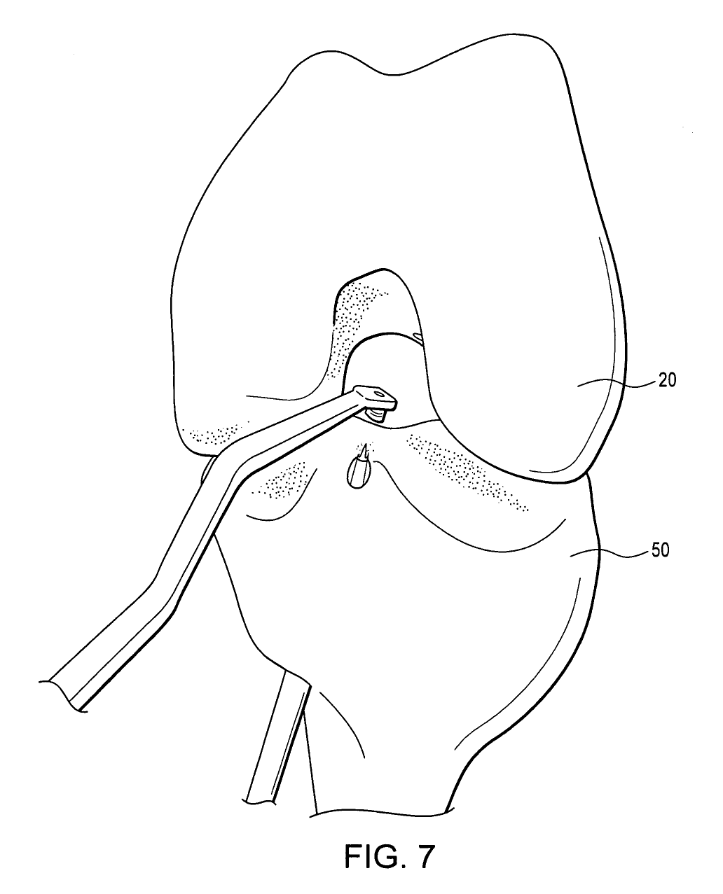

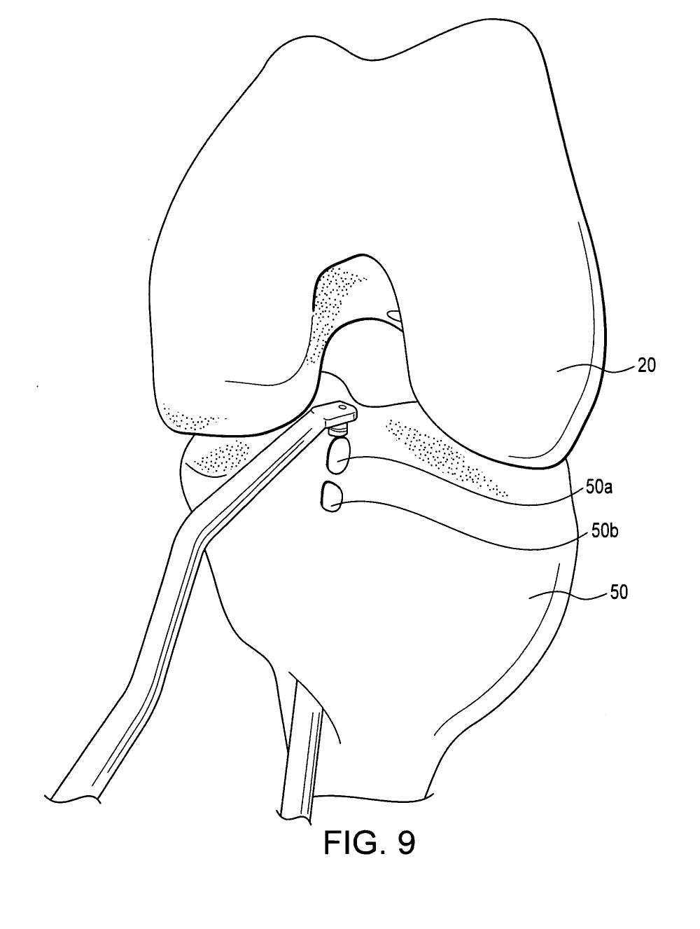

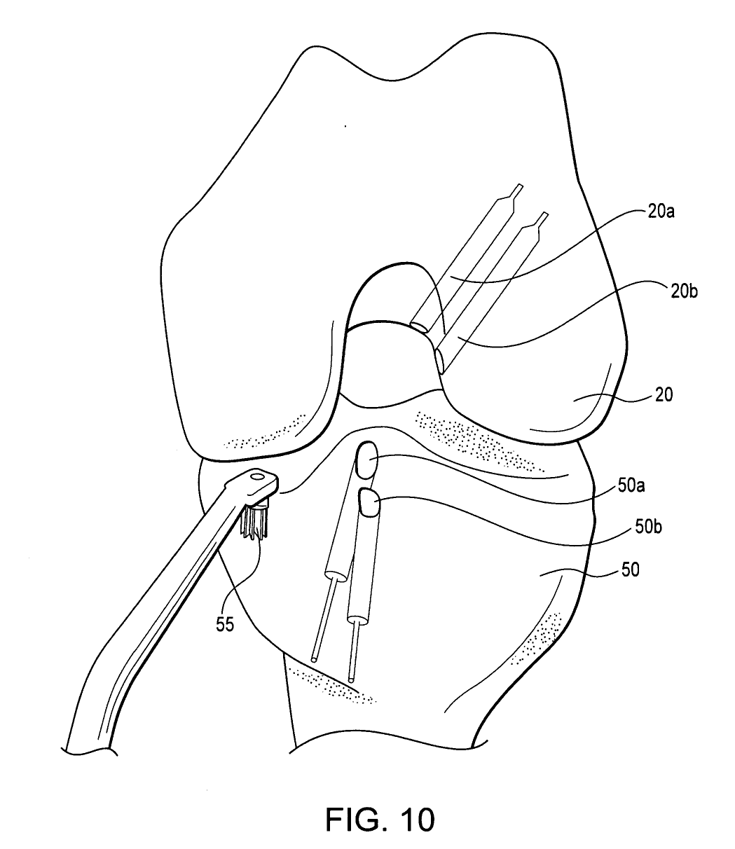

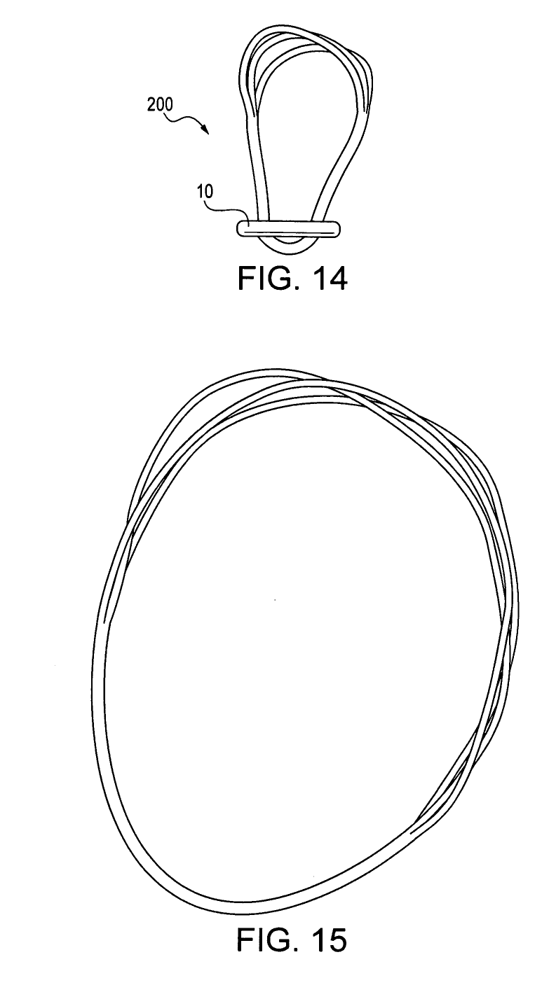

Referring now to the drawings, where like elements are designated by like reference numerals, FIGS. 1-13 illustrate various steps of an “all-inside double-bundle” method of ACL reconstruction according to an exemplary embodiment of the present invention. FIGS. 14 and 15 illustrate a continuous loop/button construct 200 used for fixating grafts according to the “all-inside double-bundle” technique of present invention and used during ACL reconstruction.

According to an exemplary embodiment of the present invention, an “all-inside double-bundle” method of ACL reconstruction using a continuous loop/button construct comprises, for example, the steps of: (i) drilling two tibial tunnels or sockets using a retrograde drill cutter which is inserted in a retrograde manner through the tibia; (ii) drilling two femoral sockets or tunnels; (iii) determining the length of two grafts (soft tissue grafts and/or BTB grafts) based on the entire length of the sockets plus the intraarticular space between them; and (iv) securing the grafts within the tibial tunnels (sockets) by using a continuous loop/button construct (Retrobutton).

The tibial sockets may be prepared by employing a retrograde drill device provided with a retrograde drill cutter detachable from a threaded guide pin, in the manner described in U.S. Patent Application Publication No. 2004/0199166. As described in U.S. Patent Application Publication No. 2004/0199166, a retrograde drill device for ACL reconstruction is provided with a retrograde drill cutter detachable from a threaded guide pin. The retrograde drill cutter is inserted in a retrograde manner through the tibia or femur by employing a retrograde drill guide pin provided with depth markings.

Once the femoral and tibial tunnels or sockets have been completed, graft insertion and fixation may be subsequently carried out. Preparation of the allograft may be conducted by employing a continuous loop/button construct 200 (FIGS. 14 and 15) provided with a button, preferably of titanium alloy, and a continuous loop attached to the button, as described in U.S. Ser. No. 11/889,740, filed Aug. 16, 2007, the disclosure of which is herein incorporated by reference. The button has an oblong configuration and a width that is preferably less than about 1 mm narrower than the width of the drill hole through which the button is inserted and subsequently passed through. The button is provided with an inside eyelet that allows the passage of the continuous loop. In an exemplary embodiment, the suture loop may be a single high strength suture such as FiberWire® suture, sold by Arthrex, Inc. of Naples, Fla., and described in U.S. Pat. No. 6,716,234, the disclosure of which is incorporated by reference herein. In another exemplary embodiment, the continuous loop may be formed of a plurality of suture strands configured to separate from a single strand to a plurality of strands in a continuous loop.

According to an exemplary embodiment of the present invention, each allograft (which may be a soft tissue graft) is folded in half over the loop of the button 200 and tension is applied. Subsequently, passing sutures are pulled and the graft is passed into the tibial tunnel or socket. When the graft reaches the opening of the tibial socket or tunnel on the tibial cortex, a slight popping sensation may be felt as the button exits and begins to flip horizontally on the cortex. Distal traction on the graft and release of the passing sutures facilitate complete deployment of the button. The passing suture may be removed and femoral fixation may be completed.

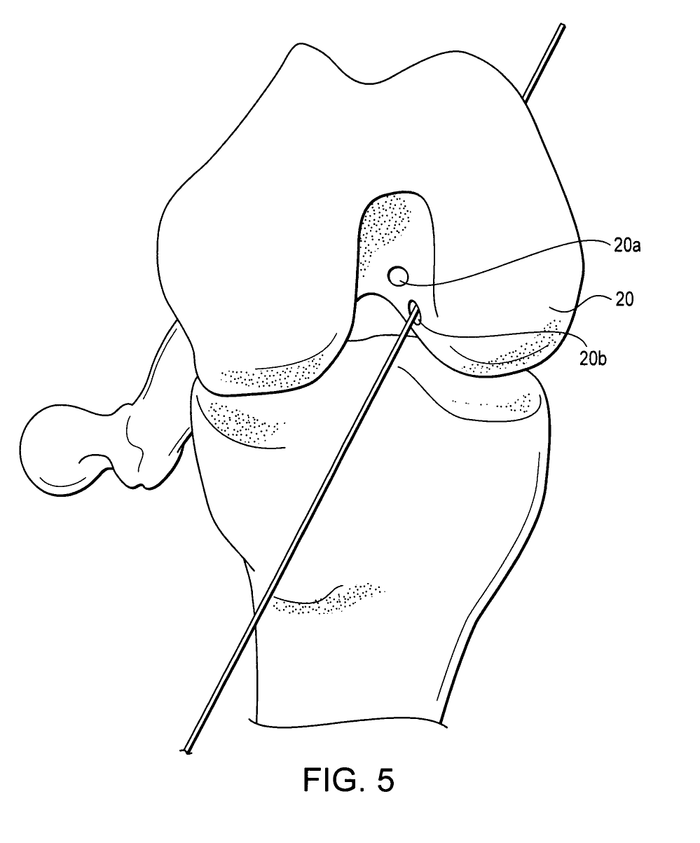

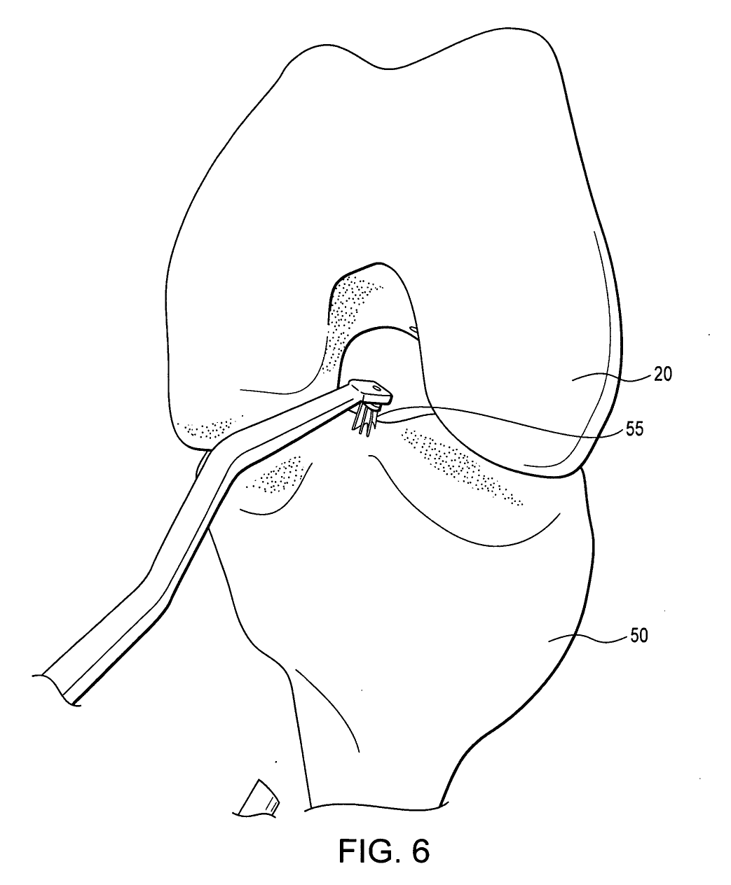

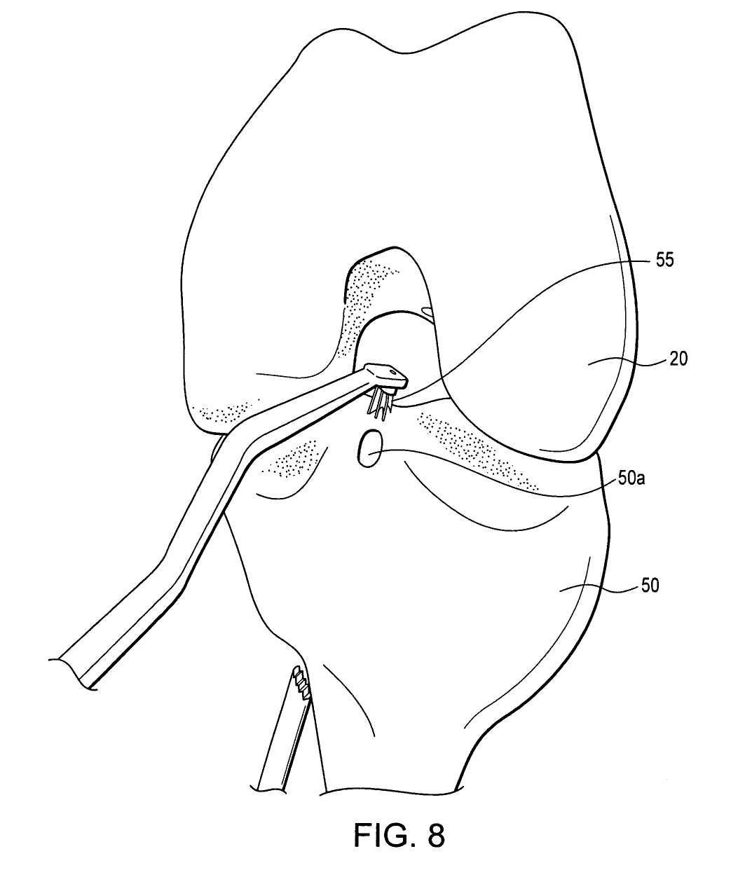

An exemplary embodiment of the present invention is described below with reference to FIGS. 1-13, which illustrate various steps of an “all-inside double-bundle” method of ACL reconstruction. As shown in FIGS. 1-5, and in accordance with an exemplary embodiment only, two femoral sockets or tunnels 20a, 20b (FIG. 5) are formed within femur 20 by either a conventional method or a retrograde method. Two tibial sockets 50a, 50b are formed in tibia 50 prior or subsequent to the formation of the femoral sockets 20a, 20b, as shown in FIGS. 6-10. The tibial sockets 50a, 50b are preferably formed using a retrograde drill cutter 55 (FIGS. 6 and 8) which is inserted in a retrograde manner through tibia 50, and as detailed in U.S. Patent Application Publication No. 2004/0199166, entitled “ACL Reconstruction Technique Using Retrodrill.”

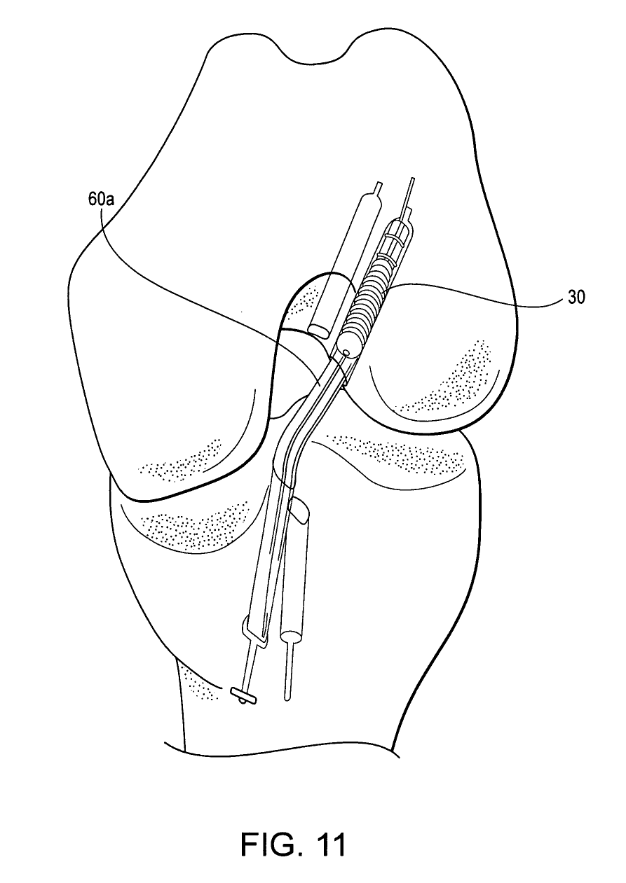

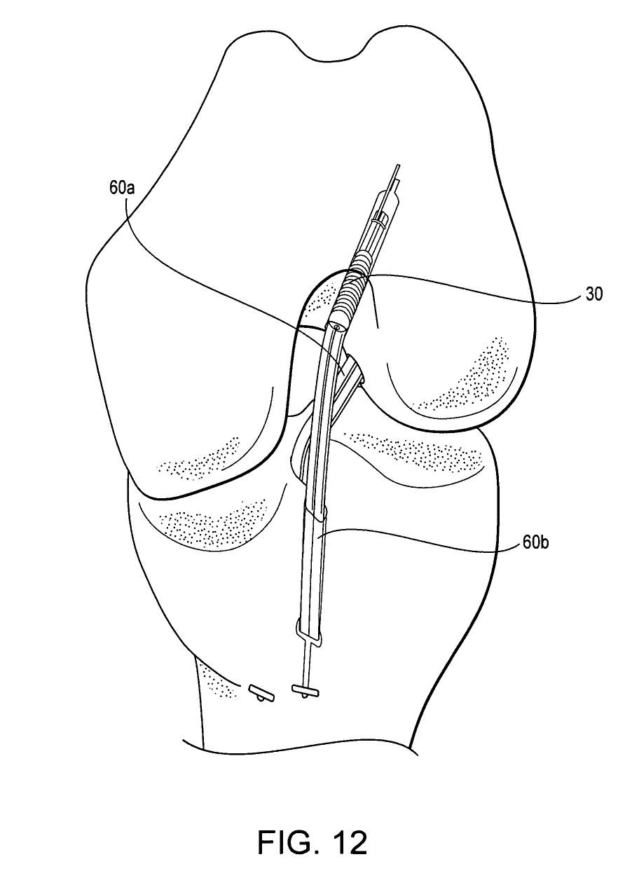

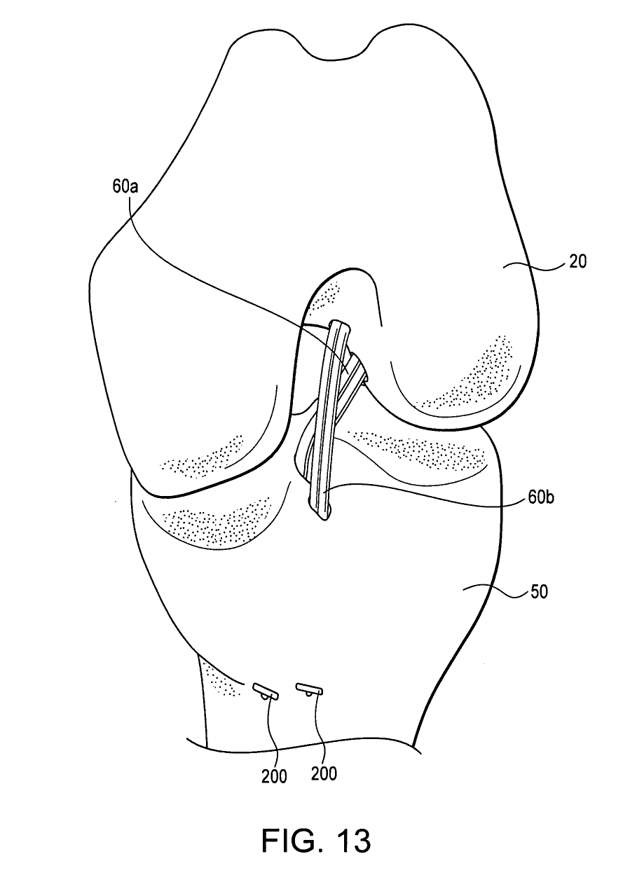

Once the two tibial sockets 55a, 55b and the two femoral tunnels 20a, 20b are formed, the length of the two individual grafts 60a, 60b (soft tissue grafts and/or BTB grafts) that will be secured within the tibial and femoral sockets is determined based on the entire length of the sockets plus the intraarticular space between them. The selected grafts 60a, 60b are then secured within the tibial tunnels (sockets) 50a, 50b by using the continuous loop/button construct 200 of FIGS. 14 and 15. The other ends of the grafts 60a, 60b may be secured within the femoral sockets 20a, 20b by employing an interference fixation device such as interference screw 30 illustrated in FIGS. 11 and 12. The final structure shown in FIG. 13 includes the two grafts 60a, 60b secured within tibial tunnels (sockets) 50a, 50b (formed in a retrograde manner) by using two continuous loop/button constructs 200, and within femoral sockets 20a, 20b by using two interference fixation devices such as two interference screws 30.

The all-inside double-bundle, quad socket technique of the present invention is more reproducible, allows individual tensioning of the AM and PL bundles at various flexion angles, addresses previous tibial fixation concerns with two screws, and provides greater graft-to-tunnel wall contact to accelerate graft incorporation than double-bundle single socket techniques. Also, the small (e.g., 6 mm) tunnels are completely filled with bone after one year post operatively. The 6 mm femoral sockets are more posterior and anatomically placed than one larger tunnel.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A method of conducting arthroscopic surgery, comprising the steps of:
forming a first plurality of bone sockets in a first bone;
forming a second plurality of bone sockets in a second bone, the first bone articulating in a predetermined manner with the second bone;
providing a plurality of grafts in the vicinity of the first and second bones;
providing a suture loop/button construct having a button with at least one eyelet and a continuous suture loop attached to the eyelet;
attaching at least one of the plurality of grafts to the suture loop/button construct; and
subsequently, positioning the graft attached to the suture loop/button construct within one of the first and second plurality of bone sockets.

2. The method of claim 1, wherein the step of attaching the graft further comprises folding the graft over the continuous suture loop.

3. The method of claim 1 further comprising the steps of:
pulling the suture loop/button construct with the attached graft through one of the first and second plurality of bone sockets;
positioning the attached graft within the first and second plurality of bone sockets; and
securing the button on a surface of one of the first and second bones.

4. The method of claim 1, wherein the step of forming the first plurality of bone sockets in the first bone includes the step of drilling the first bone, in a retrograde manner, using a rotary drill cutter.

5. The method of claim 4, wherein the rotary drill cutter is a dual-sided rotary drill cutter.

6. The method of claim 4, wherein the rotary drill cutter comprises two opposed sides and is provided with cutting surfaces on both sides, such that the rotary drill cutter is configured for cutting in two directions.

7. The method of claim 1, wherein the step of forming the second plurality of bone sockets in the second bone includes the step of drilling the second bone, in a retrograde manner, using a rotary drill cutter.

8. The method of claim 7, wherein the rotary drill cutter is a dual-sided rotary drill cutter.

9. The method of claim 7, wherein the rotary drill cutter comprises two opposed sides and is provided with cutting surfaces on both sides, such that the rotary drill cutter is configured for cutting in two directions.

10. The method of claim 1, wherein the graft is biological or non-biological tissue.

11. The method of claim 1, wherein the graft is selected from the group consisting of ligament, tendon, bone and cartilage.

12. The method of claim 1, wherein the graft is soft tissue graft or BTB graft.

13. The method of claim 1, wherein the first bone is femur and the second bone is tibia.

14. A method of ACL reconstruction, comprising the steps of:
forming a first femoral socket and a second femoral socket within the femur;
forming a first tibial socket and a second tibial socket within the tibia, wherein at least one of the first and second tibial socket is formed by drilling the tibia, in a retrograde manner, using a rotary drill cutter;
providing a first graft and a second graft in the vicinity of the femur and tibia; and
securing one end of the first and second grafts within the first and second femoral sockets and the other end of the first and second grafts within the first and second tibial sockets.

15. The method of claim 14, further comprising:
providing a suture loop/button construct in the vicinity of the femur and tibia, the suture loop/button construct comprising a button having at least one eyelet, and a continuous suture loop attached to the eyelet;
attaching at least one of the first and second grafts to the suture loop/button construct; and
subsequently securing the attached graft within one of the first and second tibial sockets.

16. The method of claim 15, wherein the suture loop is formed of a suture material comprising ultrahigh molecular weight polyethylene.

17. The method of claim 15, wherein the button has an oblong configuration.

18. The method of claim 15, wherein the button has a length of about 10 to about 20 mm.

19. The method of claim 15, wherein the button has a width that is less than about 1 mm narrower than a width of the first and second tibial sockets.

20. The method of claim 14, wherein at least one of the first and second grafts is soft tissue graft or BTB graft.

21. The method of claim 14, wherein the rotary drill cutter is a dual-sided rotary drill cutter.

22. The method of claim 14, wherein the rotary drill cutter comprises two opposed sides and is provided with cutting surfaces on both sides, such that the rotary drill cutter is configured for cutting in two directions.

23. A method of double ligament reconstruction, comprising the steps of:
forming a first plurality of sockets in a first bone by conducting a first action using a rotary drill cutter, the first bone articulating in a predetermined manner with a second bone;
forming a second plurality of sockets in the second bone by conducting a second action using the rotary drill cutter;
providing a first suture loop/button construct in the vicinity of the first and second plurality of sockets, the first suture loop/button construct comprising a button with at least one eyelet and a continuous suture loop attached to the at least one eyelet;
attaching a first graft to the suture loop of the first suture loop/button construct;
pulling the first suture loop/button construct with the attached first graft through one of the first plurality of sockets;
securing the button of the first suture loop/button construct to a bone cortex abutting the first plurality of sockets; and
securing the first graft within one of the second plurality of sockets.

24. The method of claim 23, further comprising the steps of:
attaching a second graft to a suture loop of a second suture loop/button construct;
pulling the second suture loop/button construct with the attached second graft through another of the first plurality of sockets;
securing the button of the second suture loop/button construct to a bone cortex abutting the first plurality of sockets; and
securing the second graft within another of the second plurality of sockets.

25. The method of claim 23, wherein the first bone is tibia and the second bone is femur.

26. The method of claim 23, wherein the first bone is femur and the second bone is tibia.

27. The method of claim 23, wherein the button comprises at least one opening configured to allow the suture loop to pass through it.

28. The method of claim 23, wherein the suture loop is a continuous suture loop.

29. The method of claim 23, wherein the suture loop is formed of a suture material comprising ultrahigh molecular weight polyethylene.

30. The method of claim 23, wherein the button has an oblong configuration.

31. The method of claim 23, wherein the button has a length of about 10 to about 20 mm.

32. A tissue repair assembly, comprising:
a first tissue extending through a first femoral socket and a first tibial socket, the first tissue being attached to a first button comprising a body and two eyelets extending through opposing surfaces of the body, and a continuous suture loop attached to the two eyelets; and
a second tissue in the vicinity of the first tissue, the second tissue extending through a second femoral socket and a second tibial socket.

33. The tissue repair assembly of claim 32, wherein the second tissue is attached to a second button comprising a body and two eyelets extending through opposing surfaces of the body, and a continuous suture loop attached to the two eyelets.

34. The tissue repair assembly of claim 32, wherein the first tissue is biological or non-biological tissue.

35. The tissue repair assembly of claim 32, wherein the first tissue is selected from the group consisting of ligament, tendon, bone and cartilage.

36. The tissue repair assembly of claim 32, wherein the first tissue is soft tissue graft or BTB graft.

37. The tissue repair assembly of claim 32, wherein the second tissue is biological or non-biological tissue.

38. The tissue repair assembly of claim 32, wherein the second tissue is selected from the group consisting of ligament, tendon, bone and cartilage.

39. The tissue repair assembly of claim 32, wherein the second tissue is soft tissue graft or BTB graft.
